(19) United States
(12) Patent Application Publication
(10) Pub. No.: US 2005/0187593 A1
(43) Pub. Date: Aug. 25, 2005

(54) Implantable medical device system with communication link to home appliances

(75) Inventors: Craig M. Housworth, Woodbury, MN (US); Nancy P. Pool, El Dorado Hills, CA (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/784,521

(22) Filed: Feb. 23, 2004

(51) Int. Cl. A61N 1/08

(52) U.S. Cl. 607/60

(57) ABSTRACT

An implantable medical device (IMD) system is provided including an IMD capable of generating uplink telemetry transmissions, an external medical device (EMD), such as a patient programmer or home monitor, for receiving uplink telemetry transmissions from the IMD and for transmitting data to a home electronic appliance configured to receive data transmissions from the external device. The external device is equipped with a communication interface for establishing a communication link with the home appliance, equipped with a compatible communication interface for receiving transmitted data. The home appliance may be an audiovisual appliance, personal computer or accessory, or personal communication appliance. The home appliance responds to a received transmission by any of: generating a display of transmitted data, generating a printed record of transmitted data, generating an electronic storage file, emitting an auditory or visual warning; or initiating a network transfer of data.

### CROSS REFERENCE TO PRIOR APPLICATION

This application is a continuation-in-part of prior application Ser. No. XX/XXX,XXX, filed Feb. 9, 2004, entitled “IMPLANTABLE MEDICAL DEVICE PROGRAMMER MODULE FOR USE WITH EXISTING CLINICAL INSTRUMENTATION”, which is a continuation-in-part of prior application Ser. No. 09/745,112, filed Dec. 20, 2000 entitled “INSTRUMENTATION AND SOFTWARE FOR REMOTE MONITORING AND PROGRAMMING OF IMPLANTABLE MEDICAL DEVICES (IMDs)”.

### FIELD OF THE INVENTION

The present invention relates generally to implantable medical device systems and more particularly to an implantable medical device system enabled for communication with home appliances.

### BACKGROUND OF THE INVENTION

Electronic data recording capabilities were introduced into implantable medical devices such as pacemakers, defibrillators, cardiac monitors, insulin pumps, and the like, after low power digital memory became implemented in such devices. As the storage-to-size ratio of digital memory has increased with improved technology, the total data storage capacity has improved in small implantable medical devices capable of recording physiological signals such as the ECG, blood pressure, oxygen saturation, body motion, respiration, blood flow, blood insulin concentration, etc. However, without the use of data compression methods, data storage is still limited to relatively short periods of time. The recordation of physiological data can be highly valuable to a clinician in diagnosing a patient condition, monitoring therapy effectiveness, recognizing early symptoms of a worsening condition making early intervention possible or even understanding a disease process. Therefore, in order to maintain the small size of implantable devices, it is desirable to uplink and store recorded data obtained by an implanted medical device on a periodic basis such that the limited memory capacity within the implanted device may be cleared and made available for storing more data.

As communications technologies advance, medical device systems are taking advantage of communication networks for transferring data from a remote location to a clinical center. The Medtronic CareLink™ Network, for example, allows a patient to transfer data from his or her implanted device to a home monitor unit connected to standard phone line for Internet transmission to the Carelink Network. Medical personnel at the patient's clinical center are able to review data stored in the implanted device which previously would have required an office visit to uplink stored data to an external programmer. Examples of remote monitoring medical device systems are generally described in U.S. Pat. No. 6,418,346 issued to Nelson et al., and U.S. Pat. No. 6,497,655 issued to Linberg et al., both of which patents are incorporated herein by reference in their entirety.

Remote monitoring using Internet or other network data transfer often requires the patient to actively uplink data to the monitor and initiate the transfer. In many cases, it is desirable that a patient need not intervene to initiate the transfer of data or a warning message. For example, a patient may be experiencing asymptomatic physiological changes that would be of interest to a clinician. In other cases, a patient may experience symptoms that physically disable the patient from initiating a data transfer. If large amounts of data are desired, frequent uplinking and transferring of data may become an inconvenient task to the patient. In other cases, the patient may be completely unaware of a device-related event that may require medical attention such as unexpected battery depletion or medical lead issues. Thus, it is desirable that data be transferable from an implantable medical device to an external device for data storage or for generating a warning signal for the patient or a clinician without requiring intervention by the patient. A passive communication scheme between one or more external instruments communicable with one or more implanted medical devices is generally disclosed in U.S. Pat. No. 6,574,511, issued to Lee, incorporated herein by reference in its entirety.

The use of home electronic appliances such as televisions, personal computers and associated accessories such as printers and CD-burners, video cassette or DVD recorders, cellular telephones, personal digital assistants, has become common place. In addition, many homes are equipped with an Internet connection via a hard-wired modem, or with increasing popularity, wireless connections. Furthermore, many home appliances are equipped with wireless communication interfaces such as radio frequency, infrared or ultrasound communication interfaces.

### BRIEF SUMMARY OF THE INVENTION

The availability of such appliances in the home and the associated communications technology for networking such devices provides an opportunity for interfacing implanted medical device systems with home appliances for transferring data and/or warning signals. The ability to store large amounts of physiological data over longer periods of time may be achieved by enabling the passive transfer of data to a home appliance for storage in a printed or electronic format or for immediate transfer to a central database. Warning signals may be transferred to a home appliance and delivered to the patient or to a clinical center via a home appliance using communication links already present. Through the use of home appliances in conjunction with a medical device system, the data acquisition capacity of an implanted device may be effectively extended and a patient or clinician may be quickly alerted to conditions warranting medical attention.

The present invention provides an implantable medical device system including an implantable medical device (IMD) capable of generating uplink telemetry transmissions, an external medical device (EMD), such as a patient programmer or home monitor, for receiving uplink telemetry transmission from the IMD and transmitting data to a home appliance configured to receive data transmissions from the external device. The implanted device and the external device are provided with telemetry circuitry to enable bi-directional telemetry transmissions between the implanted device and the external device. The external device is further equipped with a communication interface for establishing a communication link with a home appliance, equipped with a compatible communication interface for receiving transmitted signals or data.

The transmission of data from the external device to the home appliance may occur via a hard-wired or wireless communication link between the external device and the home appliance. The transmission may use wireless communication such as RF, infrared, or ultrasound transmission to cause a home appliance to respond to transmitted data. Network communication technology standards may be utilized for ensuring a smooth transfer of data and applications from the external device to the home appliance. Representative communication technologies include Jini, Bluetooth and HAVi.

The home appliance will respond to a received transmission from the external device. The home appliance may be any of a number of electronic home appliances including home audiovisual appliances, personal computers and accessories, and personal communication appliances. Upon receipt of the transmitted data, and depending on the type of home appliance being used, the home appliance will respond by generating a display of transmitted data, generating a printed record of transmitted data, generating an electronic storage file, emitting an auditory or visual warning; or initiating a network transfer of data.

### BRIEF DESCRIPTION OF THE DRAWINGS

FIG. 1 is an illustration of an IMD in telemetric communication with an external device having a communication link with a home appliance in accordance with the present invention.

FIG. 2 is a block diagram of the external device and home appliance shown in FIG. 1 having a communication link.

FIG. 3 is a block diagram depicting an external medical device having a communication link with home audio/visual equipment.

FIG. 4 is a block diagram depicting an external medical device having a communication link with home personal computing equipment.

FIG. 5 is a block diagram depicting an external medical device having a communication link with personal communication equipment.

FIG. 6 is a flow chart summarizing steps included in a method for transferring data from an IMD to a home appliance via an external medical device.

FIG. 7 is a flow chart summarizing steps that may be included in a method for uplinking data to an external device and transferring data to a home appliance in response to a detected cardiac arrhythmia by an IMD.

FIG. 8 is flow chart summarizing steps that may be included in another embodiment of the present invention wherein an IMD in the form of an implantable insulin delivery pump uplinks data periodically to an external device for transfer to a home appliance.

FIG. 9 is a flow chart summarizing steps included in a method by which an external medical device may request a service to be performed by a home appliance.

FIG. 10 is a schematic illustration of a general implementation of communication network technologies in an external medical device to enable communication with a home appliance.

FIGS. 11A through 11F illustrate a number of variations of communication network technology implementations that may be made for establishing a communication link between an external medical device and a home appliance.

FIG. 11G is a block diagram illustrating a communication network technology implementation that may be made for establishing a communication link between an IMD and a home appliance.

### DETAILED DESCRIPTION OF THE PREFERRED EMBODIMENTS

The present invention is directed toward providing a communication link between a home appliance and an external device having bi-directional communication with an IMD. An external device, which may be a home monitor, patient programmer or the like, is provided with a communication interface for transmitting data or signals to a compatible communications interface provided on the home appliance. Data or signal transmission from the EMD to the home appliance allows data retrieved by the EMD from an IMD to be displayed, stored, or transferred to a central database. Warning signals generated by either the IMD or the EMD based on device- or patient-related data may be displayed to the patient or transferred to a clinical center.

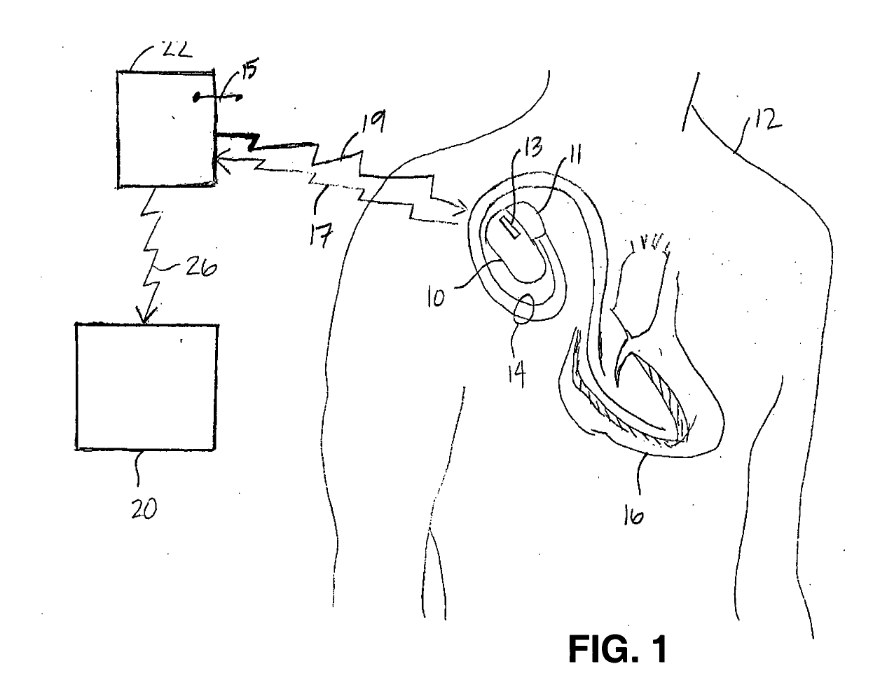

The operations performed by the home appliance (an information appliance) in response to receiving a transmission of data from the external device will depend on the type of home appliance with which the IMD system is used. One of ordinary skill in the art will recognize that numerous functions available from a variety of home appliances may be beneficial when used in conjunction with an IMD system equipped to enable a communications link with a home appliance. Home appliances appropriate for use with an IMD system may include, but are not limited to: electronic audiovisual devices such as televisions, video cassette or DVD recorders, and stereos; personal computing devices and associated auxiliary devices such as personal computers, personal digital assistants, printers disk drives, modems and monitors; and personal communication devices such as cellular telephones and fax machines. FIG. 1 is an illustration of an IMD in telemetric communication with an external programmer having a communication link with a home appliance in accordance with the present invention. IMD 10 is shown implanted in the body of a patient 12. The present invention may be implemented for use with a variety of programmable IMDs, including cardiac stimulation devices, cardiac or other physiological monitoring devices, neuromuscular stimulators, implantable drug pumps, or the like. For the sake of illustration, IMD 10 is shown here as a cardiac stimulation device coupled to a set of leads 14 used for positioning electrodes and optionally other physiological sensors in operative relation to the patient's heart 16. Leads 14 are coupled to IMD 10 via a connector block 11. Exemplary cardiac stimulation or monitoring devices with which the present invention may be employed are disclosed in U.S. Pat. No. 5,545,186 issued to Olson, et al., U.S. Pat. No. 5,987,352 issued to Klein et al., and U.S. Pat. No. 6,438,408 issued to Mulligan et al., all of which patents are incorporated herein by reference in their entirety.

IMD 10 contains an operating system that may employ a microcomputer or a digital state machine for timing cardiac sensing and stimulation functions in accordance with a programmed operating mode. The IMD 10 also contains sense amplifiers for detecting cardiac signals, patient activity sensors or other physiologic sensors for sensing the need for cardiac output, and pulse generating output circuits for delivering cardiac stimulation pulses to at least one chamber of the heart 16 under control of the operating system in a manner well known in the prior art. The operating system includes memory registers or RAM for storing a variety of programmed-in operating mode and parameter values that are used by the operating system. The memory registers or RAM may also be used for storing data compiled from sensed cardiac activity and/or relating to device operating history or sensed physiologic parameters for telemetry out on receipt of a retrieval or interrogation instruction. All of these functions and operations are well known in the art, and many are employed in other programmable IMDs to store operating commands and data for controlling device operation and for later retrieval to diagnose device function or patient condition.

IMD 10 is in telemetric communication with external device 22. Exemplary external devices that may be located in a patient's home in which the present invention may be implemented to achieve communication between the device a home appliance are disclosed in U.S. Pat. No. 6,647,229 issued to Bourget and U.S. Pat. No. 6,249,703, issued to Stanton, et al., both of which patents are incorporated herein by reference in their entirety. Programming commands or data are transmitted between an IMD RF telemetry antenna 13 and an external RF telemetry antenna 15 associated with the external device 22. The external RF telemetry antenna 15 may be contained in a programmer RF head so that it can be located close to the patient's skin overlying the IMD 10. Such programmer RF heads are well known in the art. See for example U.S. Pat. No. 4,550,370 issued to Baker, incorporated herein by reference in its entirety. The external device 22 may be designed to universally program IMDs that employ conventional ferrite core, wire coil, RF telemetry antennas known in the prior art and therefore also have a conventional programmer RF head and associated software for selective use with such IMDs.

Alternatively, the external RF telemetry antenna 15 can be located on the case of the external device 22, and the external device 22 can be located some distance away from the patient 12. For example, RF telemetry antenna 15 may be integrated with external device 22 and external device 22 may be located a few meters or so away from the patient 12 and utilize long-range telemetry systems. With regard to the present invention, such long-range telemetry systems are preferable over systems requiring an RF head such that passive telemetry transmission may occur for successively initiating signal transmissions from EMD 22 to a home appliance 20 without patient interaction. Long-range telemetry systems would allow bi-directional communication between IMD 10 and external device 22 when IMD 10 is within a communication range of external device 22. Moreover, the patient 12 may be active, e.g., partaking in normal household activities or exercising during an uplink telemetry interrogation of real time ECG or physiologic parameters. Telemetry systems that do not require the use of a programmer RF head are generally disclosed in U.S. Pat. No. 6,240,317 Villaseca et al., U.S. Pat. No. 6,169,925 issued to Villaseca et al., and U.S. Pat. No. 6,482,154, issued to Haubrich et al., all of which patents are incorporated herein by reference in their entirety.

In an uplink telemetry transmission 17, the external RF telemetry antenna 15 operates as a telemetry receiver antenna, and the IMD RF telemetry antenna 13 operates as a telemetry transmitter antenna. Conversely, in a downlink telemetry transmission 19, the external RF telemetry antenna 15 operates as a telemetry transmitter antenna, and the IMD RF telemetry antenna 13 operates as a telemetry receiver antenna. Both RF telemetry antennas are coupled to a transceiver comprising a transmitter and a receiver.

In accordance with the present invention, external device 22 is adapted for generating signal transmissions to be received by a home appliance 20. External device 22 and home appliance 20 are equipped with compatible communication interfaces that may be hardwired or wireless. Preferably, a standard wireless communication link is provided to avoid electrical isolation issues associated with a hardwired link.

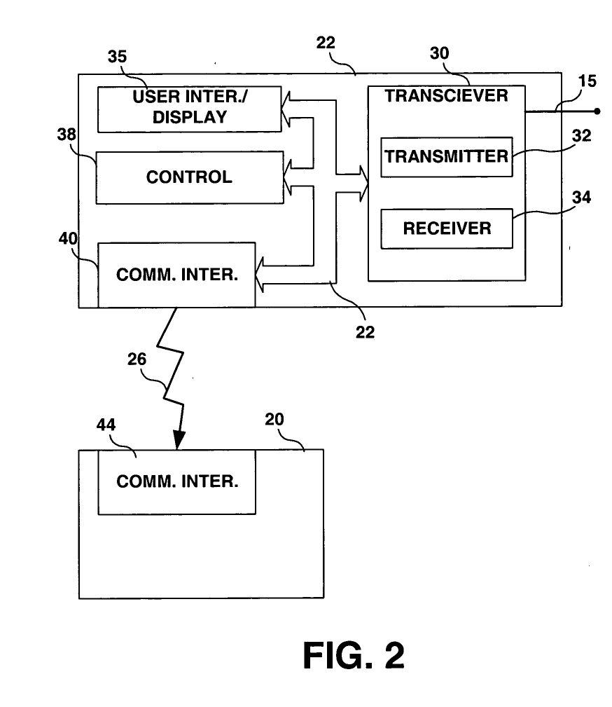

FIG. 2 is a block diagram of the external device and home appliance shown in FIG. 1 having a communication link. As shown in FIG. 2, external device 22 may be configured as a programmer having a transceiver 30 including a transmitter 32 and receiver 34 for bi-directional communication with IMD 10. External device 22 will typically include a user interface and display 35 and a central control system 38, which may be in the form of a microprocessor and associated memory. External device 22 may be embodied as a patient programmer and, as such, include push buttons and LEDs or sound emitting elements to function as a user interface and display. In other embodiments, the user interface and display may include a touch pad or screen, a graphical user interface, or other interface and display elements known for use with home patient programmer or monitoring devices. Control system 38 controls the interrogation and transmission functions of device 22 via transceiver 30 in response to user-entered functions. Control system 38, transceiver 30, and user interface/display are coupled via a data bus 22.

In accordance with the present invention, external device 22 is further equipped with a communications interface 40 to allow external device 22 to allow transmission of data from external device 22 to home appliance 20. A communication link 26 is established between communication interface 40 of EMD 22 and a compatible communication interface 44 provided on home appliance 20. Communication interfaces 40 and 44 may be designed to transmit and receive data via RF, ultrasound, or infrared transmissions. For example, communication link 26 may be established between communication interfaces 40 and 44 using commercially available wireless technology such as Bluetooth or WiFi, or Digital European Cordless Telecommunications (DECT). A home appliance 20 may be manufactured with an interface compatible with these commercially available wireless technology formats pre-installed or may be configured with an auxiliary device including communication interface 44 to enable communication link 26 with external device 22.

Communication link 26 may be a unidirectional communication link such that EMD 22 transmits a signal or data to be received by home appliance 20. In some embodiments, communication link 26 may be configured for bi-directional communication such that signals may be transmitted from home appliance 20 to EMD 22. Such signals that may be transmitted from home appliance 20 to EMD 22 may include, for example, signals confirming a complete transmission has been received or confirming the results of a test routine run to establish communication link 26.

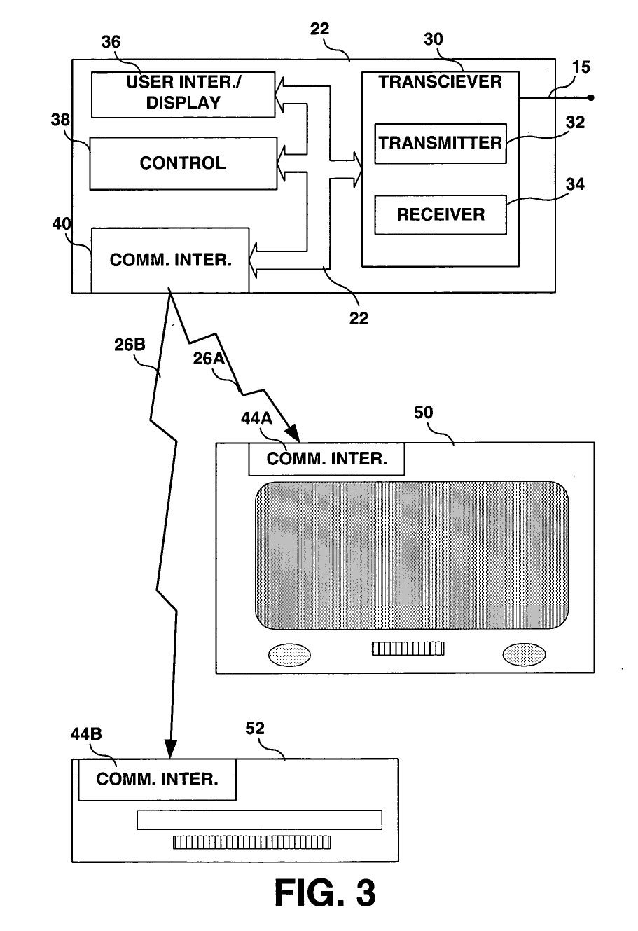

FIG. 3 is a block diagram depicting external device 22 having a communication link 26 with home audio/visual equipment. A television 50 and/or video recorder 52 may be equipped with communication interfaces 44A and 44B, respectively, for receiving data transmission from external device 22 via communication link 26A or 26B, respectively. Television 50 may respond to a data transmission by displaying a visual or audio message. For example, a patient warning signal may be displayed by television 50 upon receipt from external device 22. Recorder 52 may respond to a data transmission by recording transmitted data to data storage medium such as a video cassette or DVD. Other types of home audio/visual equipment may alternatively be provided with a communication interface for establishing a communication link with external device 22, such as a stereo receiver, CD player, and the like.

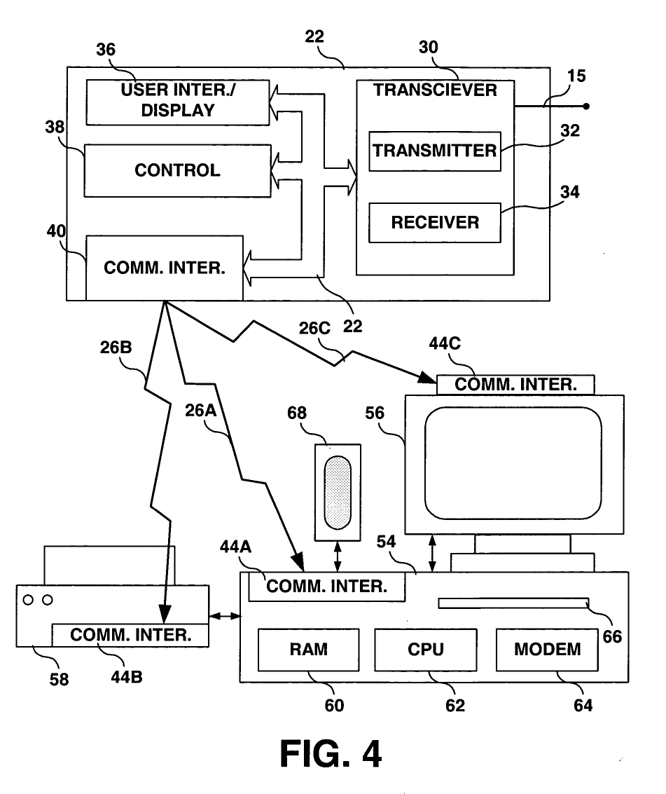

FIG. 4 is a block diagram depicting external device 22 having a communication link with home personal computing equipment. A personal computer 54 is shown including RAM 60, a central processing unit (CPU) 62, a modem 64, and a disk drive 66. Personal computer 54 may be coupled to auxiliary devices such as monitor 56, printer 58 and speaker 68. Personal computer 54 is shown equipped with a communication interface 44A configured for receiving data transmissions from EMD communication interface 40 via communication link 26A.

Personal computer 54 responds to received data from external device 22 by executing a data storage, display, or transfer operation. Received data may be stored in RAM 60, written to an electronic data storage medium by disk drive 66, or transferred to printer 58 to produce a written record. Received data may additionally or alternatively be used to generate a patient warning signal displayed on monitor 56 or broadcast by speaker 68 and/or initiate an Internet transfer of data to a central data base at the patient's clinical center. A clinician may then review transferred data at any time or be alerted of a warning message regarding a device-related or patient-related condition.

Furthermore, received data may undergo processing and analysis according to software programs loaded into computer 54 and executed by CPU 62. The results of such analysis may then be stored, transferred, printed, displayed, or cause a warning signal to be generated and displayed on monitor 56 or speaker 66, printed on printer 58, or transferred via modem 64.

In alternative embodiments, auxiliary devices associated with home computer systems may be equipped with communication interfaces for establishing a communication link directly with external device 22. As shown in FIG. 4, printer 58 may be equipped with a communication interface 44B to receive data transmissions from external device 22 and respond thereto by producing a printed record of received data. In another example, monitor 56 may be equipped with a communication interface 44C and upon receipt of data transmissions from external device 22 generate a display of received data. Other types of auxiliary devices known for use with personal computing systems, such as CD burners, speakers, and the like, may be equipped with a communication interface for establishing a communication link with external device 22 and respond to a received data transmission by storing, displaying, or transferring received data.

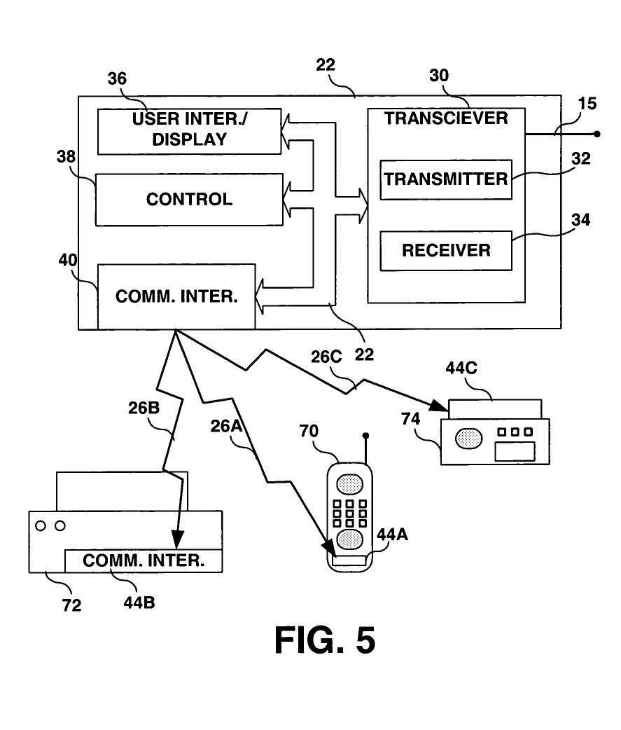

FIG. 5 is a block diagram depicting external device 22 having a communication link with personal communication equipment. Various personal communication equipment such as a cellular telephone 70, fax machine 72, or answering machine 74, may be equipped with a communication interface 44A, 44B or 44C, respectively, for establishing a communication link 26A, 26B and 26C, respectively, with external device 22. A fax machine 72 may respond to a data transmission by transmitting a facsimile data record to a clinical center. Data transmission to cellular telephone 70 may cause a wireless transmission to be placed, either to the patient's phone 70 or to a clinical center to deliver a warning. Data transmission to answering machine 74 may generate a recorded message to alert the patient to a warning message.

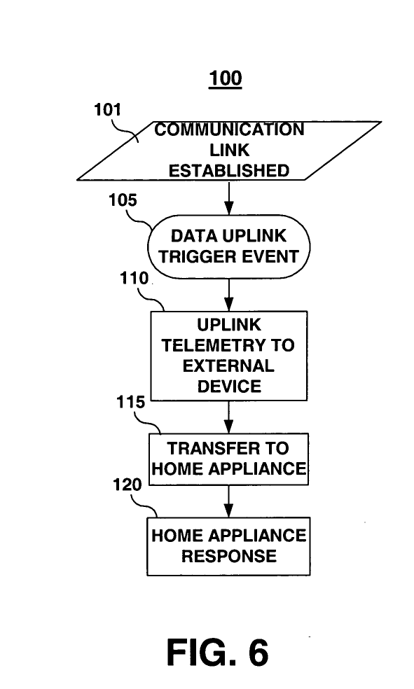

Any of a variety of home appliances, including electronic devices, audio/visual electronics, home computing system elements, or home communications devices, may be configured for receiving and responding to data transmissions from an EMD. Of course, the present invention is not limited to the specific devices or types of devices given as examples herein. FIG. 6 is a flow chart summarizing steps included in a method for transferring data from an IMD to a home appliance via an EMD.

An initialization step 101 is performed upon installing external device 22 in the home along with any available home appliances to be used in conjunction with the medical device system. At step 101, communication link 26 is established between external device 22 and a home appliance 20 by verifying any necessary connections between external device 22 and home appliance 20 and running test routines to verify a proper communication link 26. External device 22 thereafter “knows” which home appliances are available for transferring data such that when a data transfer becomes necessary, a communication link 26 is already established and available. Alternatively, when a data transfer becomes necessary, external device 22 may utilize test routines to determine which home appliances are available via a communication link and thereby establish a communication link 26 on an “as needed” basis.

Execution of the remaining steps of method 100 is initiated at step 105 when a data transfer triggering event is recognized by IMD 10. In response to the triggering event, data is uplinked from IMD 10 to the external device 22 at step 110. A data transfer triggering event may be an interrogation command received by IMD 10 from external device 22. An interrogation command may be delivered manually by a user using external device 22. For example, a patient may deliver an interrogation command when feeling symptomatic or on a scheduled basis according to clinician instructions.

Alternatively, an interrogation command may be generated by external device 22 whenever it is within communication range with IMD 10. Using long-range telemetry an IMD may be within communication distance of an external device when the patient and external device are within a home environment. When external device 22 is within communication range of IMD 10 and a telemetry link is established, external device 22 may automatically initiate a telemetry uplink transmission, without user intervention. A passive communication scheme between an external instrument communicable with an IMD is generally disclosed in U.S. Pat. No. 6,574,511 to Lee, incorporated herein by reference in its entirety. As will be described in greater detail below, uplinked telemetry obtained by passive interrogation of IMD 10 by external device 22 may undergo analysis by external device 22 to determine if additional actions are needed, i.e., a data transfer to a home appliance for permanent storage, transfer to a clinical center, or generation of a warning signal.

In other embodiments, a data uplink trigger event may be generated within IMD 10 according to device-related or patient-related conditions detected by IMD 10. For example, IMD 10 may perform diagnostic tests to evaluate lead status and if a lead-related issue is identified, a data uplink from IMD 10 to external device 22 may be triggered to occur as soon as IMD 10 is within a communication range with external device 22. Other device- or patient-related conditions that may be recognized by IMD 10 and trigger a data uplink to occur include, but are not limited to, battery status, a change in pacing threshold, a detected arrhythmia, or a change in a monitored physiological signal. Thus, data may be uplinked to external device 22 automatically when a medically relevant device- or patient-related event is recognized by IMD 10, without intervention by a patient or other user.

A data uplink trigger event may also be scheduled to occur on a regular basis so as to effectively extend the physiological data storage capacity of the IMD. Data stored within the IMD may be uplinked to an external device for transfer to a home appliance for permanent storage or transfer to a clinical database, allowing occupied memory within IMD 10 to be cleared and made available for storing new data. Physiological data may thus be stored without interruption due to full memory within IMD 10 and without over-writing older data that would otherwise be lost. A data uplink trigger event for transferring stored physiological data may be generated either by external device 22 or IMD 10 on a programmable periodic basis or by IMD 10 when available memory for storing physiological data has reached a certain capacity. Physiological data may then be uplinked to external device 22 as soon as IMD 10 is within communication range of external device 22.

Once an uplink telemetry transmission is completed at step 110, external device 22 may execute a data transfer via communication link 26 to a home appliance 20 at step 115. External device 22 may transfer a signal or data to one or more home appliances depending on the type of signal or data to be transferred and the home appliances available. Likewise, depending on the type of signal or data transferred and the type of home appliance receiving the transmission, the home appliance will respond to the received transmission at step 120, e.g. by storing, transferring, printing or analyzing data or generating a warning signal as has been described previously in conjunction with FIGS. 2 through 5.

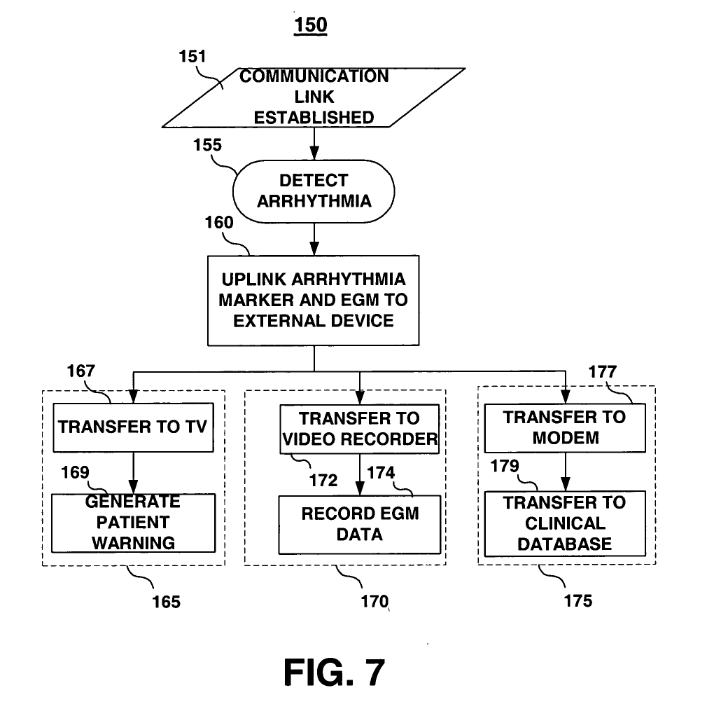

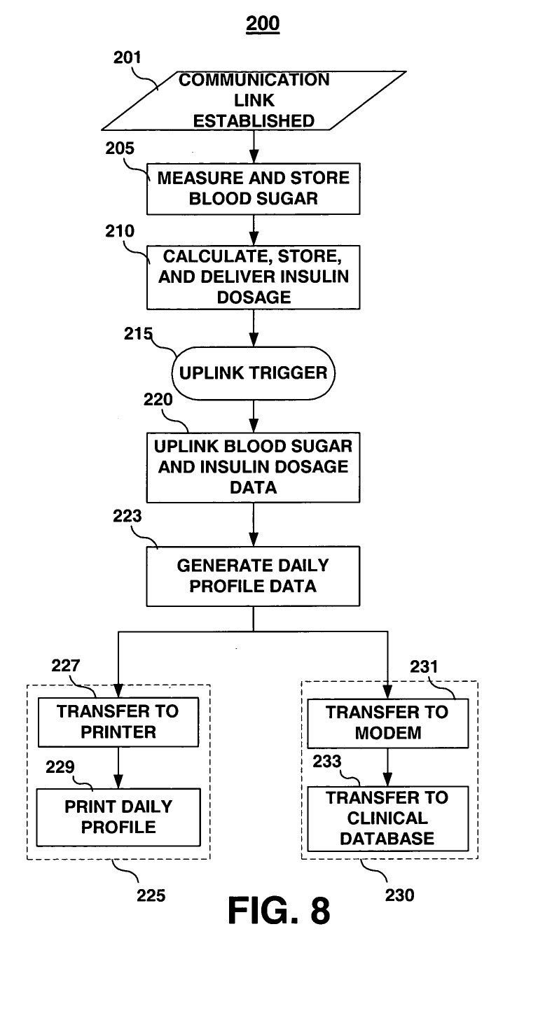

It is to be understood that multiple data uplink triggering events may be selected to be enabled such that an uplink transmission may occur in response to a number of different predetermined conditions. Likewise, an EMD may in turn initiate a data or signal transfer to one or more home appliances upon receiving uplink telemetry, and thereby by initiate multiple responses by the one or more home appliances. Examples of methods for transferring data from an EMD to multiple home appliances to initiate multiple possible responses are described below in conjunction with FIGS. 7 and 8.

FIG. 7 is a flow chart summarizing steps that may be included in a method for uplinking data to an external device and transferring data to a home appliance in response to a detected cardiac arrhythmia by an IMD. Method 150 includes an initiation step for establishing communication links between an external device 22 and a number of home appliances, in this example, a television, a video recorder and a modem having Internet access. At step 155, the IMD detects an arrhythmia which is designated as an uplink triggering event recognized by the IMD. Hence, at step 160 data is uplinked from the IMD to external device 22. Preferably an arrhythmia event marker and real time EGM signal are uplinked to the external device.

External device 22 may then initiate a number of subroutines 165, 170 and 175 depending on the type and severity of arrhythmia detected. Each subroutine includes at least a step for transferring a signal or data to a selected home appliance and a step for executing a desired response or service by the home appliance. Subroutine 165 includes transferring a signal from external device 22 to a television at step 167. The television responds to the received transmission at step 169 by broadcasting a patient warning. The patient warning may be a video or audio signal alerting the patient and/or household members that a potentially serious arrhythmia has been detected so that appropriate emergency actions may be quickly taken. In one example, the television may respond to an arrhythmia warning signal received from the external device with a sudden volume increase.

Subroutine 170 includes transferring real-time EGM data from external device 22 to a video recorder at step 172. The video recorder responds to the transferred signal by recording real-time EGM data to a cassette or DVD at step 170 with appropriate time and data labels.

Subroutine 175 includes transferring data to a modem at step 177. The modem responds by transferring the data, which may include both an arrhythmia event warning and real time EGM data to a clinical database via the Internet. The IP address for the patient database is stored in the external device and a special VPN client is installed to allow safe access to the patient database via the modem. The data transferred to the clinical database is available for immediate review by a clinician and may be used to initiate an emergency response.

Thus, it is recognized that external device 22 may be equipped with a number of subroutines for initiating data transfer to and a response from a home appliance. Subroutines that may be installed and called upon will depend on the type of data uplinked from the IMD, results of analyses that the external device may perform on uplinked data, and the types of home appliances available. It is recognized that, depending on the particular IMD implanted and its function(s), numerous subroutines may be conceived of which involve transferring data to any of a number of home appliances for triggering a response by the home appliance in accordance with the functions available from the home appliance.

FIG. 8 is flow chart summarizing steps that may be included in another embodiment of the present invention wherein an IMD in the form of an implantable insulin delivery pump uplinks data periodically to an external device for transfer to a home appliance. Method 200 includes an initiation step 201 for establishing a communication link between an external device and a home appliance as described previously. Steps 205 and 210 are performed by the IMD in accordance with normal device operation. At step 205, the IMD measures the blood sugar concentration and stores the result in IMD memory. At step 210, the IMD computes the appropriate insulin dosage, records the result in IMD memory, and delivers the dosage to the patient. An exemplary implantable system and method for obtaining a measure of a patient's insulin demand and blood glucose level is generally disclosed in U.S. Pat. No. 6,261,280 issued to Houben et al., incorporated herein by reference in its entirety.

At step 215 an uplink triggering event occurs. The uplink triggering event initiates an uplink telemetry transmission of the stored blood sugar and insulin dosage data at step 220. As described previously, the uplink triggering event may be generated by the IMD or by the external device. An uplink triggering event generated by the IMD may occur with every blood sugar measurement and insulin dosage calculation, after a given number of blood sugar measurements and dosage calculations, after the available memory has reached a certain capacity limit, or on a periodic time interval. Alternatively, an uplink triggering event may be generated in response to an interrogation command from the external device automatically delivered on a periodic basis. e.g., a daily basis. If the IMD and external device remain within a communication range, uplink transmission of blood sugar concentration and insulin dosage data may occur continuously as it is collected.

Once the external device has received an uplink transmission at step 220, additional processing or analysis of received data may be performed at step 223. For example, data representing a daily profile may be generated to allow a graphical or tabular display of the data. The external device may then call upon one or more subroutines for transmitting a signal or data to a home appliance and thereby initiate an operation by the home appliance. Examples of possible subroutines are shown in FIG. 8.

Subroutine 225 includes a step 227 for transmitting daily profile data to a printer, and a step 229 wherein the daily profile data is printed by the printer. Subroutine 230 includes a step 231 for transmitting daily profile data to a modem, and a step 233 wherein the daily profile data is transferred to a clinical database via the modem and an Internet connection. As noted previously, a number of subroutines are conceivable which include the transmission of a signal or data from an external device to a home appliance depending on the type of data uplinked from the IMD, results of any analysis performed by the external device on the uplinked data, and the home appliances available.

The communication link established between an external medical device and a home appliance may rely on any available communication connection technologies, such as Bluetooth, or Jini headers on Java applications, for ensuring compatible data transmission using any available network interfaces, including hardwired or wireless interfaces such as infrared or RF network interfaces. By enabling the EMD to transfer a signal to a home appliance the EMD is able to request the home appliance to provide a desired service.

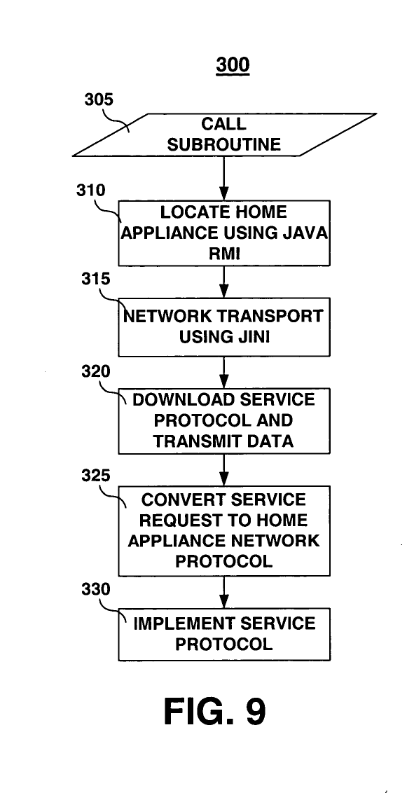

FIG. 9 is a flow chart summarizing steps included in a method by which an external medical device may request a service to be performed by a home appliance. At step 305, an external medical device calls upon a subroutine for transmitting data to a home appliance and requesting a service to be performed by the home appliance. Some examples of subroutines that may be called upon were described previously in conjunction with FIGS. 7 and 8 and may include requesting a printing operation, a data storage operation, a display of transmitted data or a warning signal, or a data transfer to a central database.

After calling upon a subroutine, the external medical device locates an available home appliance capable of the service required by the subroutine using the communication technology implemented in association with the communication interface. In the example shown in FIG. 9, the home appliance is located by the external device using Jini technology, which employs Java Remote Method Invocation (RMI™). Network transport via the network interfaces provided on the external device and the home appliance at step 315 is accomplished using Jini technology. Jini technology allows any network protocol supported by the operating systems of the external device and the home appliance to be used.

After the home appliance is located and network transport established, the external device downloads the service application and transmits related data to the home appliance by running Java code supplied by the subroutine. If necessary, the service request is converted by Jini technology into a protocol usable by the home appliance at step 325. At step 330, the transmitted code and data are used by the service protocol of the home appliance to execute the requested service.

Thus, by utilizing available communication connection technologies, such as Jini or Bluetooth, service protocols available in a variety of home appliances may be requested by an external medical device, regardless of the type of network interface used or the operating systems installed in the appliance and the external device.

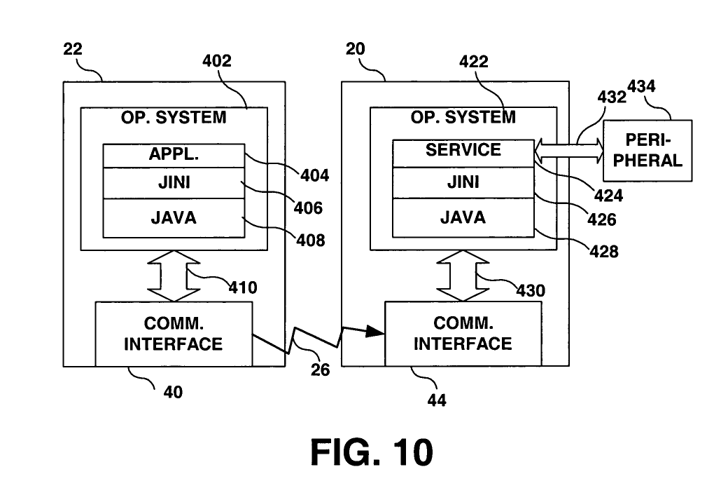

FIG. 10 is a schematic illustration of an implementation of Jini technology in an external medical device having communication with a home appliance. The external device 22 requires the service of home appliance 20. A subroutine for calling upon home appliance 20 to provide a service is stored as an application 404 in external device 22. Application 404 is written in Java language 408 having a Jini header 406. The operating system 402 of external device 22 implements an appropriate network transport 410 via a communication interface 40 to establish communication link 26 with home appliance 20.

Likewise, home appliance 20 includes a communication interface 44 for receiving a transmission from external device 22. The operating system 422 of home appliance 20 implements a network transport 430 for uploading a transmitted service request and associated data. A service protocol 424 is written in Java language 428 having a Jini header 426. The home appliance 20 may then execute the service protocol 424 or call upon a peripheral device 434 using a separate device/bridge protocol 432 for performing the requested service. Communication link 26 may be established over a number of network architectures including the Internet, a local area network (LAN), a wide area network (WAN), a wireless communication network, community access television (CATV) network, public switched telephone network (PSTN), the integrated services digital network (ISDN).

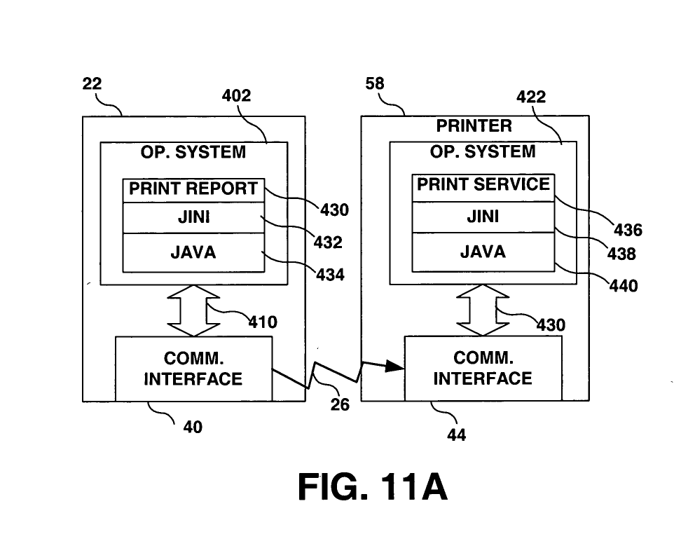

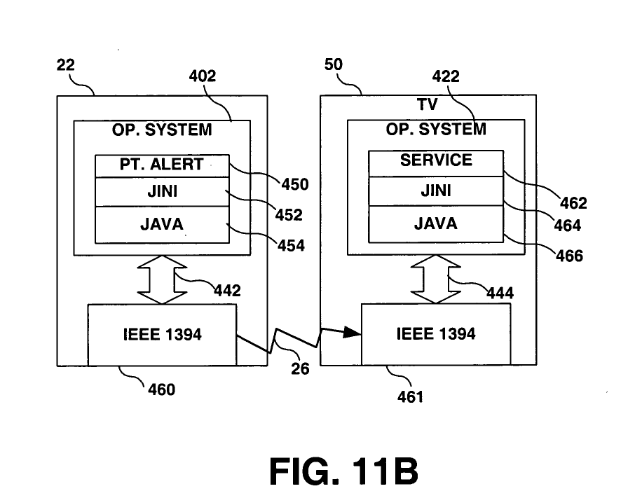

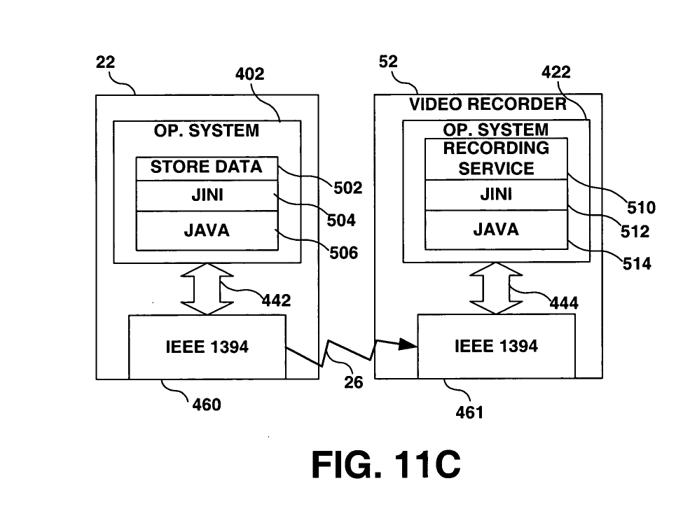

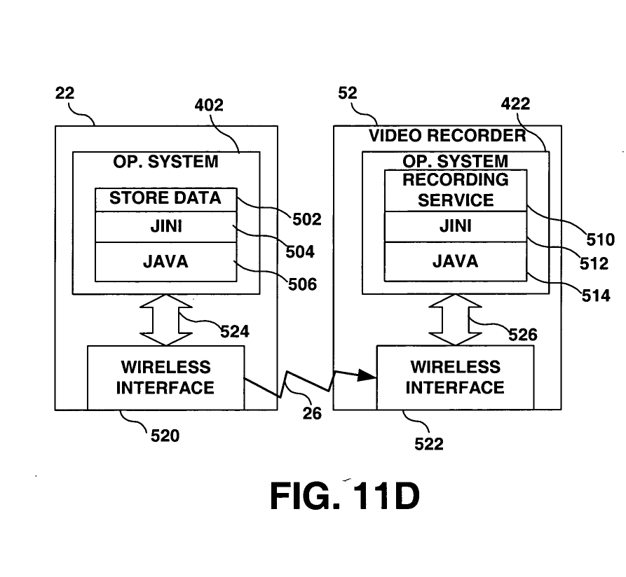

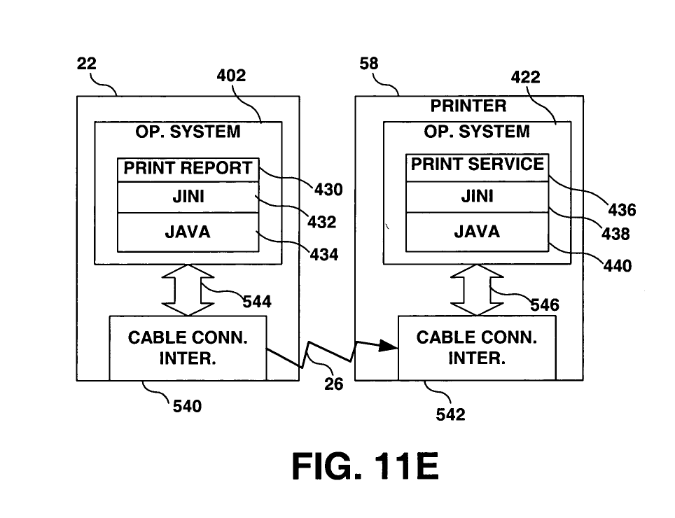

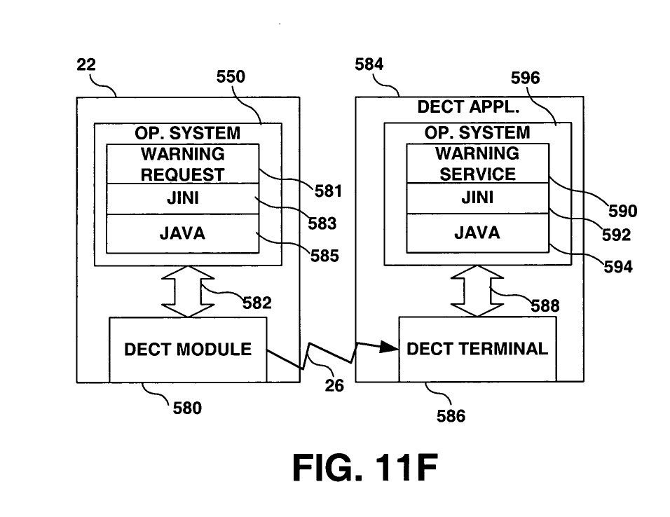

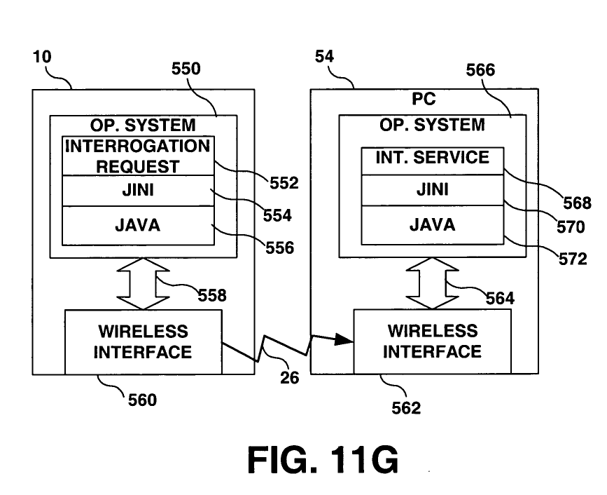

FIGS. 11A through 11F illustrate a number of variations of communication network technology implementations that may be made for establishing a communication link between an external medical device and a home appliance. In FIG. 11A, an external medical device 22 is provided with a network communication link with a printer 58. An application 430 written in Java 434 with a Jini header 432 stored in external device 22 may be called upon by the operating system 402 for generating a printed report of patient or device-related data. The operating system 402 invokes a network transport 410 utilizing a communication interface 40 which provides a communication link 26 with communication interface 44 of printer 58. Communication interfaces 40 and 44 may be implemented according to any available network technology specification such as an Ethernet connection using TCP/IP, infrared connection using IrDA, or wireless connection using Bluetooth. Printer 58 responds to the transferred application by invoking a printing service protocol 436, also written in Java language 440 with a Jini header 438.

FIG. 11B is a schematic diagram illustrating an external medical device 22 having a communication link with a home audiovisual appliance, such as a television 50. In this case, operating system 402 of external medical device 22 may invoke an application 450 written in Java 454 with a Jini header 452 for generating a display warning on television 50. External medical device 22 is configured with an IEEE 1394 interface 460 which is used in the home audiovisual appliance industry to allow interoperatibility of consumer electronic devices using the HAVi standard specification. When the patient alert application 450 is activated, operating system 402 utilizes the HAVi network 442 to establish a communication link 26 with television 50, which in turn invokes a service protocol 462 written in Java 466 having a Jini header 464. Television 50 may then broadcast the patient warning via an audio of visual signal.

In a similar arrangement, an external medical device 22 may be provided with a HAVi network communication link 26 with a video recorder 52 as shown in FIG. 11C. Each of the external device 22 and video recorder 52 are provided with IEEE 1394 interfaces 460 and 461, respectively, to establish communication link 26 using the HAVi standard network transport 442 and 444, respectively. In this example, an application 502 may be called upon by operating system 402 of external device 22 to initiate data recording by video recorder 52. Video recorder 52 responds to a transmitted service request by invoking a recording service protocol 510 for recording data, which may be written in Java language 514 with a Jini header 512.

Alternatively, external device 22 and video recorder 52 may be equipped with wireless communication interfaces 520 and 522, respectively as shown in FIG. 11D. A network transport standard 524 and 526, such as Bluetooth, for use with wireless networks may then be utilized once the video recorder 52 is located external device 22 using Jini technology. Other technology, like Piano, can be built on top of Bluetooth to specify the type of information that may be exchanged between external device 22 and video recorder 52.

FIG. 11E illustrates yet another implementation of communication technology between an external device and a printer. In the embodiment shown, a JetSend protocol (developed by Hewlett-Packard) allows external device 22 to exchange information with printer 58 using a common data format after Jini technology has been used to connect the two devices. Thus, operating system 402 of external device 22 utilizes a Jetsend protocol 544 to transfer information via a cable connection interface 540 to a compatible cable connection interface 542 of printer 58 which also uses a JetSend protocol 546.

In FIG. 11F, EMD 22 is enabled as a DECT device having been provided with a DECT module 580 for establishing a wireless communication link 26 with a DECT terminal 586 for communicating via network transport protocol 588 with a DECT appliance 584. DECT appliance 584 may be a telephone or telefax machine. Operating system 550 of EMD 22 may execute a warning request application 581, written in Java 585 with a Jini header 581, upon receiving a telemetry transmission from an IMD indicating a device- or patient-related condition warranting medical attention. For example, a warning signal for a detected arrhythmia or hypoglycemic state or other potentially serious physiological condition may be requested.

The warning request is transferred via the appropriate network protocol 582 and DECT module 580 to a DECT appliance 584, which in turn initiates a warning service 590. The warning service 590 may execute a specific telephone call to all DECT devices available in the home to warn household members that the IMD has detected a potentially serious condition. Upon receiving the warning signal, all the DECT devices may issue an alarm ring. The warning service may include printing an event report on a DECT telefax. The warning service may additionally or alternatively issue a telephone call via an ISDN communication network to a clinical database and transfer data to the database.

The various embodiments described herein have been directed toward providing a communication link between an EMD and a home appliance. However, it is conceivable that a communication link may be established between an IMD and a home appliance using wireless communication technologies. FIG. 11G is a diagram of one possible embodiment wherein an IMD 10 is provided with a wireless communication interface 560 for establishing a communication link 26 with a compatible communication interface 562 of a home appliance, in this example a personal computer (PC) 54. An application program 552 may be written in Java 556 with a Jini header 554 for requesting an interrogation service 568 wherein PC 54 is requested to interrogate IMD 10 to retrieve stored or real-time patient- or device-related data.

Operating systems 550 of IMD 10 and 566 of PC 54 may employ Bluetooth or another network transport protocol standard to transport data between IMD 10 and PC 54 via wireless communication interfaces 560 and 562. Upon receiving a service request using Jini technology, PC 54 may interrogate IMD for retrieving authorized types of data.

Thus, an implantable medical device system has been described in which communication with a home electronic appliance is established to allow transfer of data or signals between the medical device system and the home appliance. One of skill in the art having the benefit of the teachings provided herein will recognize that numerous implementations are possible for establishing such a communication link with a home appliance and for generating a response by the home appliance. The various embodiments described herein, therefore, are intended to be illustrative of the concepts of the present invention and should not be considered limiting with regard to the following claims.

1. An implantable medical device system, comprising:
an implantable medical device generating uplink telemetry transmissions and receiving downlink telemetry transmissions; and
an external medical device for receiving uplink telemetry transmissions from the implantable medical device, generating downlink telemetry transmissions to the implantable medical device, and transmitting data to a home appliance, wherein the external medical device further includes a communication interface for establishing a communication link for transmitting data to a home appliance equipped with a compatible communication interface for receiving transmissions from the external medical device;
a home appliance responsive to transmission received from the external medical device.

2. The implantable medical device system according to claim 1, wherein the home appliance is an electronic audio/visual appliance including any of a television, a stereo, or a video recorder.

3. The implantable medical device system according to claim 1, wherein the home appliance is a personal computer or associated auxiliary component including any of a printer, an electronic storage medium, a modem, a monitor, a speaker, or a personal digital assistant.

4. The implantable medical device system according to claim 1, wherein the home appliance is a personal communication appliance including any of a cellular phone or a fax machine.
